(19) United States
(12) Patent Application Publication
(10) Pub. No.: US 2022/0390628 A1
(43) Pub. Date: Dec. 8, 2022

(54) PARTICLE INDUCED RADIOGRAPHY SYSTEM

(71) Applicants: Academia Sinica, Taipei City (TW); National Central University, Taoyuan City (TW)

(72) Inventors: Mythra Varun Nemallapudi, New Taipei City (TW); Chih-Hsun Lin, Taipei City (TW); Shih-Chang Lee, Taipei City (TW); Augustine Ei-fong Chen, Taichung City (TW)

(73) Assignees: Academia Sinica, Taipei City (TW); National Central University, Taichung City (TW)

(21) Appl. No.: 17/805,869

(22) Filed: Jun. 8, 2022

Related U.S. Application Data
(60) Provisional application No. 63/197,999, filed on Jun. 8, 2021.

(51) Int. Cl. G01T 1/20

(52) U.S. Cl. CPC G01T 1/20184; G01T 1/20185; G01T 1/20188

(57) ABSTRACT

The invention is related to particle induced radiography system, comprising a particle radiation source device, implant module, external detector device, central module and other controls, in which the implant module comprises active and/or passive components in tandem with the readout electronics and communication chosen to measure the beam properties and to generate and detect secondary gamma photons from the nuclear interactions, the external detector device provides a position sensitive gamma detector with a high detection efficiency, good spatial resolution and a relatively large field of view necessary for particle treatments useful in monitoring both the implanted device and the patient anatomical areas under treatment, and the external detector device can also be used to perform 3D spectral imaging on any material samples using proton beam as a probe.

### CROSS-REFERENCE TO RELATED APPLICATION

This application claims the benefit of U.S. Provisional Patent Application No. 63/197,999, filed Jun. 8, 2021, which is incorporated by reference herein in its entirety.

### FIELD OF THE INVENTION

The invention is in the field of radiation physics, in particular high energy physics. High energy particle detectors are increasingly finding applications in medical imaging especially for cancer diagnostics using gamma photons generated by dedicated tracers. With the increasing use of particle therapy for treating cancer, the detection of prompt gamma of the order of several MeV has garnered interest as a means of verifying the proton range. The invention applies the particle induced radiography techniques for range verification and imaging for biological tissue and non-biological materials.

### BACKGROUND OF THE INVENTION

Proton therapy employs high energy protons to treat cancer tumors with a high precision. However, several factors such as computed tomography (CT) conversion uncertainties, patient positioning, and patient anatomical changes etc. introduce uncertainties to the estimation of precise depth dose deposition. Monitoring the emitted secondary particles can be an indirect way of verifying the incident proton range.

However, the monitoring of real-time process is obstructed by a high radiation environment and high energy gamma photons, which can-not be collimated effectively. Also, a neutron background radiation that affects the signal and the detector adversely. As such, the detectors in proton therapy that are built for range verification have a limited applicability due to a lower efficiency and limited range.

This invention proposes a new detector design with a higher detection efficiency with an innovative design. This invention can also be useful in estimating the elemental composition and hence material changes associated with the tumor during treatment. This invention applies an indirect method relying on computation that increases the applicability of the method in a variety of treatment scenarios.

The purpose of the present invention is to make it relevant for the proton therapy where prompt gamma is emitted in relation to the beam and the target material. This will result in a successful range verification.

The purpose of the present invention is also to use the proton beam as a probe to perform gamma imaging in any target material.

### SUMMARY OF THE INVENTION

The present invention is a particle induced radiography system. The purpose of the invention is to detect precisely the location of protons within an object and detect the position distribution of a gamma source, which presents the location of target object, achieving a high level of gamma collimation while still maintaining a high detection efficiency.

Another application of the present invention is used as a 3D imaging system which can obtain the information of the interest space through the detector system coupled with a scanning pencil beam. Obtaining a 3D distribution of the prompt gamma source while the proton beam is scanned in the transverse plane. This can help us probe the elemental distribution of the target material.

### BRIEF DESCRIPTION OF THE DRAWINGS

FIG. 1 shows a full system with all the sub-components in a preferred embodiment using only the implant.

FIG. 2 shows a full system with all the sub-components in a preferred embodiment using only the external detector device.

FIG. 3 shows a full system with all the sub-components in a preferred embodiment.

FIG. 4 shows a detailed view of the implant module.

FIG. 5 shows the scintillation and detection system of the implant module.

FIG. 6 is a block diagram of the communication module.

FIG. 7 shows each sub-module designed based on the collimation requirements.

FIG. 8 shows that multiple sectors are obtained by first rotating about the beam axis and further translating along the beam axis.

FIG. 9 shows that multiple sub-modules in each sector are obtained by rotation along the azimuth.

FIG. 10 illustrates a plot of the proton count peak position vs beam energy for various range shifter densities.

FIG. 11 illustrates a plot for gamma count peak position vs beam energy for various range shifter densities.

FIG. 12 illustrates a plot of the depth dose peak position vs beam energy for various range shifter densities.

FIG. 13 is a block diagram of the central module for interfacing with the other systems.

FIG. 14 is a diagram of an example of the particle induced radiography system.

FIG. 15 is a top view of the 3D imaging system.

FIG. 16 is a side view of the 3D imaging system.

FIG. 17 is an illustration of the experimental measurement using proton induced gamma from a thin 48Ti target to verify the proton range.

FIG. 18 is a plot of Geant4/GATE simulation describing the positional resolution of a single module of the external detector-2 for different collimator gaps w1 obtained by moving a 990 keV gamma source along the measurement axis.

FIG. 19 is a plot of Geant4/GATE simulation where 40 MeV protons were irradiated on a water phantom. The plot describes the depth distribution of the 4.4 MeV gamma resulting from the 16O(p,x)12C* reaction as measured by the detector along with the true isotopic distribution obtained from the simulation.

FIG. 20 is a plot of the prompt gamma spectrum of 48Ti (FIG. 20A) irradiated by 14.52 MeV protons and iron targets (FIG. 20B) irradiated by 14.14 MeV protons. The background spectrum without any targets is shown for 13.1 MeV protons.

FIG. 21 is a plot of the experimental data describing the production cross sections of 158 keV, 309 keV and 984 keV gamma from a 48Ti target irradiated with protons of various energies.

### DETAILED DESCRIPTION OF THE INVENTION

A particle induced radiography system comprising: (a) a particle radiation source device configured to irradiate a beam; (b) a beam control device configured to adjust the particle radiation source device to control the beam energy; (c) an implant module configured to present a location of an object and to receive and detect the beam from the particle radiation source device, wherein the implant module comprises: an implant detection unit configured to detect the number of particles from the beam, at least one implanted material configured to interact with the beam that irradiated by the particle radiation source device to generate a secondary particle, an array detector configured to detect the secondary particle, an electronic and communication module _1 configured to readout data from the array detector and to output the readout data, and an extension unit configured to provide for optical or mechanical tracking of the implant module; (d) an external detector device configured to receive the data from the electronic and communication module_1 and detect the secondary particle which is generated from the implanted material to generate a signal and output to the central module for integrating; (e) a positioning module configured to obtain location of the object in order to localize the image from the implant module and the external detector device; and (f) a central module configured to process the signals, perform computation and communicate pertinent control signals, wherein the central module receives and/or transmit signals from the implant module or the external detector device and transmits to other modules.

In some embodiments, the system further of the implant module further comprises a package configured to serve as a container for the implant module.

In some embodiments, the secondary particle is gamma, electron, neutron or proton.

In some embodiments, wherein the secondary particle is gamma and it can be prompt or delayed in nature.

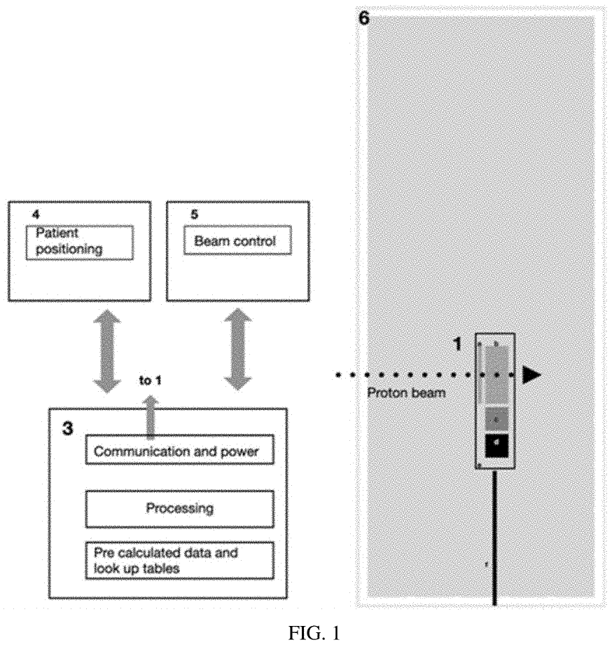

FIG. 1 shows the full system with all the sub-components in a preferred embodiment using only the implant module 1. Implant detection unit 1a is a thin silicon array detector or other thin metal array designed to detect the number of protons during the verification routine. The implant detection unit 1a measures the beam current using a direct charge collector such as silicon detector to generate an energy independent signal which will be integrated with dedicated electronics in the electronic and communication module_1 1d. The implant detection unit 1a has a maximum out-put for a narrow range of proton energies near the Bragg peak and it will enable us to identify the deviation from the beam center and readjust the position of the beam in combination with the central module 3. The central module 3 achieves this by monitoring the beam profile obtained from the pixel distribution of the implant detection unit 1a and calculating the position of the expected maxima.

Implanted material 1b interacts with the incoming proton beam to generate secondary particles (prompt gamma) that will be detected by an array detector 1c and an external detector module 2. Our cross-section measurements for some chosen materials indicates that Titanium among other materials can be clearly identified in relation to the proton energy.

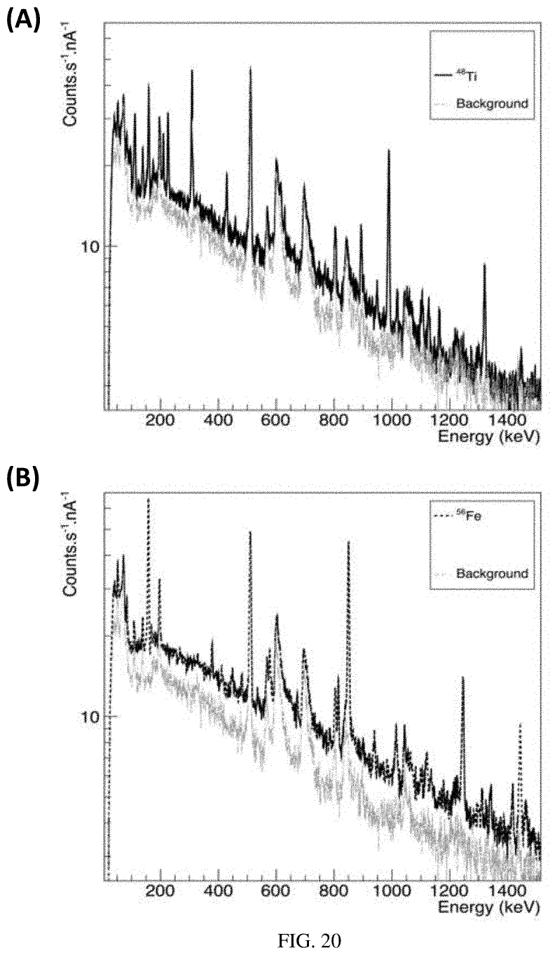

As shown in FIG. 20, the measured energy spectrum of the gamma photons emitted by 48Ti (FIG. 20A) and 56Fe (FIG. 20B) targets irradiated respectively with 14.52 MeV and 14.14 MeV protons are shown in FIG. 20. These experimental measurements were performed on 0.1 mm×50 mm×50 mm targets. Protons of 30 MeV and 15 MeV were first attenuated to lower energies before irradiating the 48 Ti target.

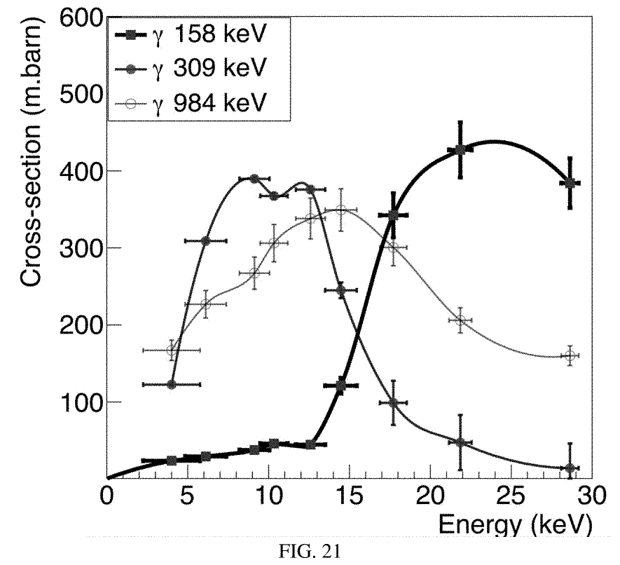

As shown in FIG. 21, the resulting values shows the production cross sections of 158 keV, 309 keV and 984 keV gamma emitted by the 48Ti target irradiated with protons of various energies.

Possible choice of materials includes Titanium, Gold and other noble elements. The shape and size of target are chosen to be cylindrical bullets with variable inner diameter, or springs or an array of thin strips. Inner hole of the hollow cylindrical target can be used to house a crystal.

An array detector 1c is an array of scintillating crystals, silicon photomultiplier, photomultiplier tube, avalanche photodiode, PMT or other optically sensitive detector thereof. In this embodiment that comprises miniaturized scintillator and SiPM array and is used to detect the secondary gamma generated by the implanted material 1b. The array specifications are chosen in order to obtain the depth distribution of the created secondaries with a high detection probability. The array detector 1c is also aimed at detecting the gamma during beam mode emitted from the positron emitting isotopes and other isotopes with shorter lifetime. This mode of detection is suitable for flash mode operation with lower doses and shorter irradiation times.

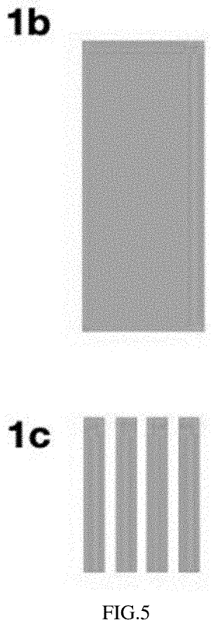

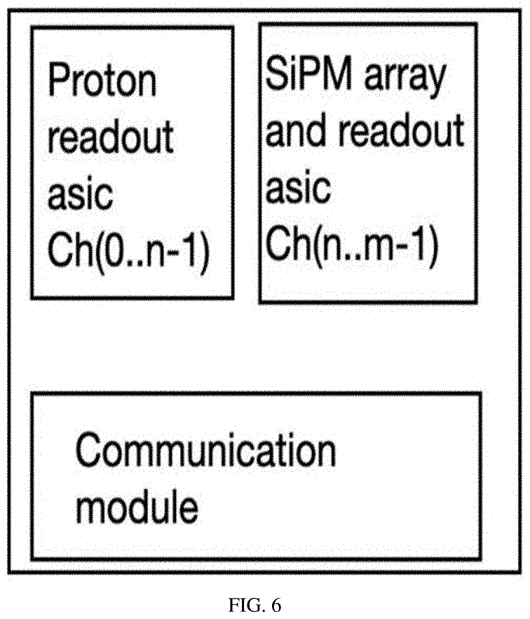

Electronic and communication module_1 1d is designed to read the proton detector output and or the SiPM detector output. 1d comprises a current integrating circuit to obtain the proton number from each pixel. The electronic and communication module_1 1d comprises a dedicated multichannel application-specific integrated circuit (asic) to integrate the current signal from each channel, digitize the information and transfer the event data with channel numbers ‘0 to n-1’ to the electronic and communication module_1 1d. The electronic and communication module_1 1d also consists of a second multichannel asic to readout the event data with channel numbers ‘n to m-1’ from the SiPM and transfers the information to the electronic and communication module_1 1d. The communication module reads the event data and transmits them wirelessly to external detector device 2. Alternatively, the events are transferred to the package 1e from which they are further transmitted wirelessly. The above description was shown in the FIG. 6.

Package 1e, is a chassis for the implant module 1 which is made from a bio safe and FDA approved material. The material for the package 1e is chosen to be non-conductive and non-corrosive. The package 1e has an electro-mechanical connector for connecting to an extension unit 1f.

Extension unit 1f is the part of the implant module 1 that can transmit the information from the implant and also contain the elements for optical or mechanical tracking of the implant. This is useful for locating the position and orientation of the implant and transmitting the values to the central module 3.

In a preferred embodiment as described in FIG. 1, the implant module 1 is the only sensitive element in the range verification system. This configuration is useful for a prostate cancer treatment scenario. Alternatively, the implant module 1 may be ingested through the esophagus while performing lung cancer surgeries. The implant module 1 may also be placed inside the oral cavity during treatments concerning the head and neck cancers.

In a preferred embodiment, the implant module 1 is placed inside the object or placed on the surface and it can be used in combination with several others to form an external wearable belt that can be mounted on the patient close to the tumor off site.

The object herein used in the specification which means patient, organ, tissue, animal, plant or other non-biological materials such as mineral, rock.

In a preferred embodiment, the implant module 1 is only a proton detector. A low dose scan is performed using proton beam with energies sufficient for the protons to reach the detector after passing through the patient. The obtained signals may be compared to the pre-calculated values to determine the range shift. This method can be a non-invasive approach for range verification using the previously described implant module 1.

In another preferred embodiment, the implant module 1 can be reduced to a compact passive material that can be directly inserted into or very close to the tumor site. The marker can be imaged using CT scan prior to the treatment to determine the relative position of the marker with respect to the tumor precisely. In this configuration an external detector device 2 is imperative to identify the marker location by detecting the characteristic gamma

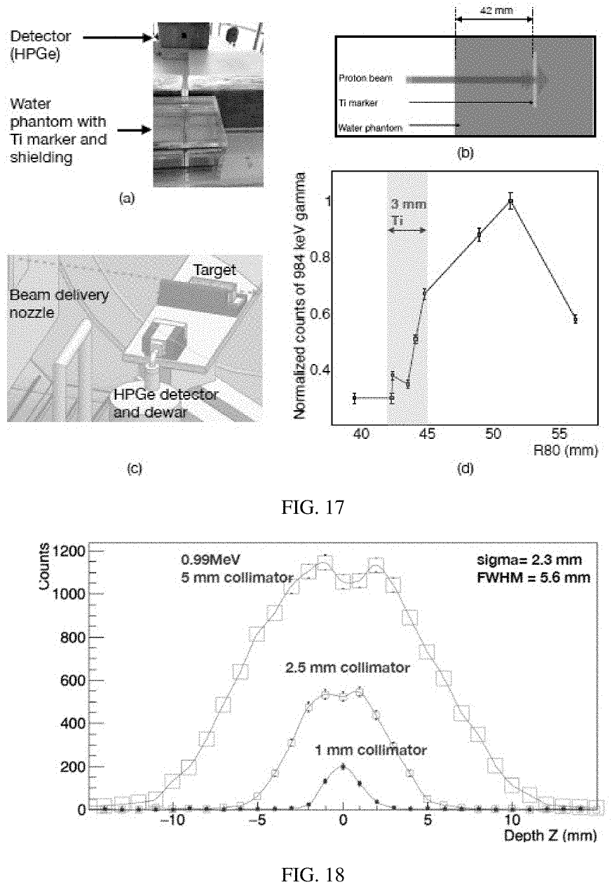

As shown in FIG. 17, one of the embodiments of the implant module 1, where in the target material when irradiated with high energy protons emits a characteristic gamma spectrum useful to validate the proton range. Illustration of the utility of a 48Ti thin implanted marker to correlate the relative intensity of the characteristic prompt gamma with the expected peak position of the depth dose at different energies. (a), (b) and (c): Experimental setup at CGMH proton therapy facility shown in different views. Proton beam was irradiated on a water phantom with an inserted Ti marker (3 mm thick) placed between 42 mm and 45 mm from the beam entrance. (d): The measured counts of 984 keV gamma line emitted by 48Ti as a function of the R80 depth (80% of the peak dose on the distal side). The gamma counts have been normalized to the incident beam current and irradiation time and the normalized values are plotted relative to the peak.

TABLE 1
Experimental settings used in the test with proton irradiation
on a water phantom with an inserted Ti marker.

| Beam energy | Beam current | R80 position | Irradiation |
|---|---|---|---|
| (MeV) | (nA) | (mm) | time (s) |
| 70 | 0.267 | 39.4 | 133 |
| 74 | 0.297 | 42.3 | 123 |
| 74.8 | 0.299 | 42.3 | 164 |
| 76.4 | 0.301 | 43.5 | 196 |
| 78 | 0.383 | 44.1 | 190 |
| 80 | 0.389 | 44.8 | 162 |
| 84 | 0.400 | 48.9 | 151 |
| 86 | 0.406 | 51.3 | 128 |
| 90 | 0.418 | 56.3 | 183 |

The table 1 corresponds to the FIG. 17 and is related to an embodiment of the implant module 1.

External Detector Device 2

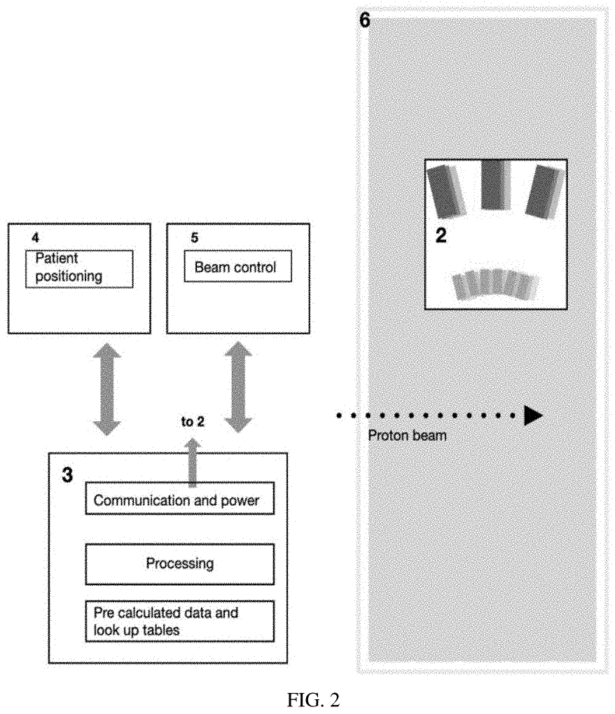

As shown in FIG. 2, the external detector device 2 comprises the sensitive array detector 2b, the readout electronics and communication module_2 2c, and the mechanical unit 2d for housing and internal control for adjusting the collimators and external positioning unit 2e for global orientation and motion. The sensitive array detector 2b in tandem with the collimators 2a is designed in order to localize the position of a detected gamma photon to the order of a few millimeters. The design aims to maximize the detection efficiency while minimizing the positional uncertainty of the gamma detected. The mechanical unit has sub-components attached to the individual elements of the crystal array in order to readjust the focusing. The mechanical unit consists of linear motion control stages to move the external detector device 2 to the position specified by the central module 3. An optional neutron detector and or proton detector can be included in the external detector device.

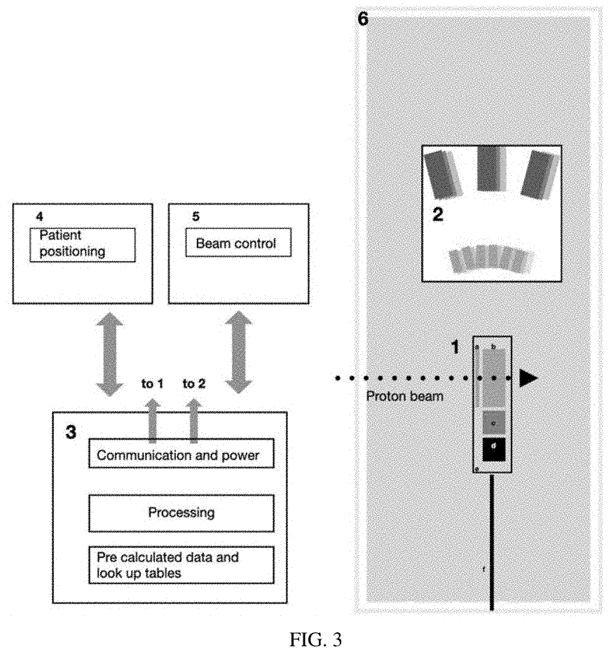

As shown in FIG. 3, the preferred embodiment of the full system comprises the implant module 1, the external detector device 2, the central module 3, along with the patient positioning unit 4 and beam control device 5. In this embodiment, the monitoring of the gamma can be performed simultaneously on the inside for observing the proton range, and on the outside for imaging the material composition of the target material.

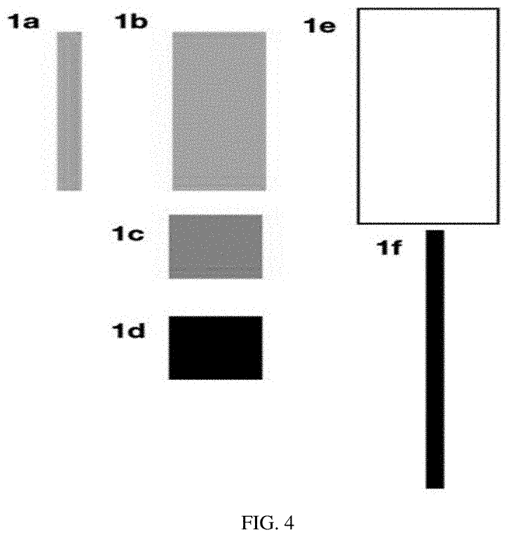

As shown in FIG. 4, the locational relationship of the implant module 1 is shown with all the sub-components separated from each other for illustration. The implant module 1 comprising an implant detection unit 1a configured to detect the number of particles from the beam, at least one implanted material 1b configured to interact with the beam that irradiated by the particle radiation source device to generate a secondary particle, an array detector 1c configured to detect the secondary particle, an electronic and communication module_1 1d configured to readout data from the array detector 1c and to output the readout data, a package 1e configured to serve as a container for the implant module 1a, and an extension unit if configured to provide for optical or mechanical tracking of the implant module 1a. Alternatively, the implant detection unit 1a, the implanted material 1b, and the array detector 1c can be implemented as a proton detector to detect the flux of protons impinging on the implant module 1.

As shown in FIG. 5, the sub-components of the implanted material 1b and the array detector 1c are shown wherein the implanted material 1b is configured to generate secondary particles, and the array detector 1c is an array of detectors comprising sensitive crystals coupled to photodetectors.

As shown in FIG. 6, the electronic and communication module_1 1d is designed to read the proton detector output and or the SiPM detector output. the electronic and communication module_1 1d comprises a current integrating circuit to obtain the proton number from each pixel. The electronic and communication module_1 1d comprises a dedicated multichannel application-specific integrated circuit (asic) to integrate the current signal from each channel, digitize the information and transfer the event data with channel numbers ‘0 to n-1’ to the electronic and communication module_1 1d. The electronic and communication module_1 1d also consists of a second multichannel asic to readout the event data with channel numbers ‘n to in-1’ from the SiPM and transfers the information to the electronic and communication module 1d. The electronic and communication module_1 1d reads the event data and transmits them wirelessly to external detector device 2. Alternatively, the events are transferred to the package 1e from which they are further transmitted wirelessly.

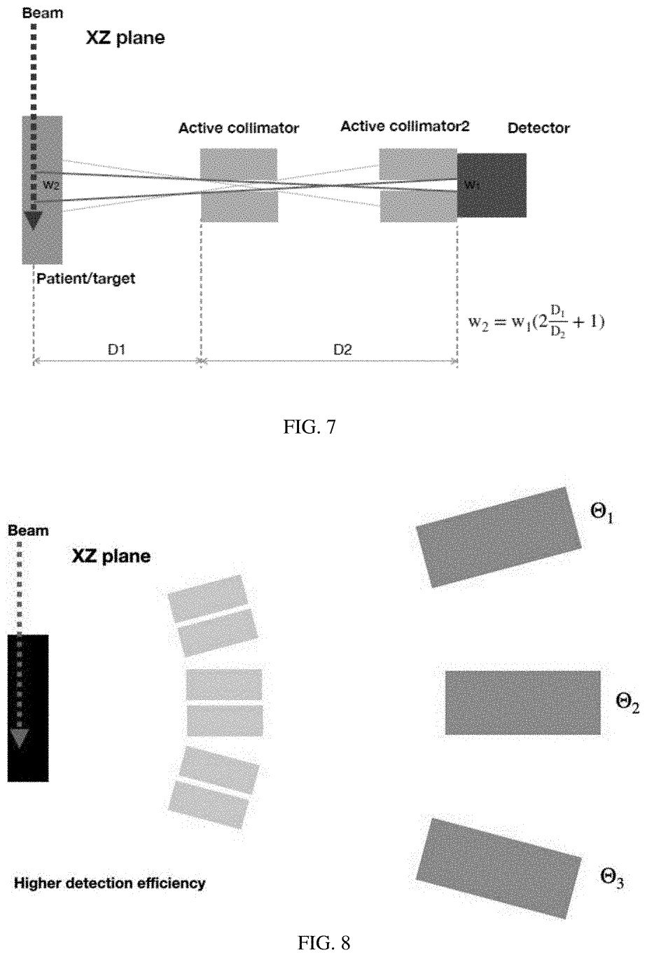

As shown in FIG. 7, the geometry of the module is described. The basic element of the external detector device 2 is a sub-module which comprises at least one collimator 2a and a sensitive array detector 2b. In one embodiment, the external detector device 2 contains multiple sub-modules within the same planar sector. In addition, the arrangement of the sub-modules in same planar sector focuses on the same spatial point. Each sector can be linearly shifted using electro-mechanical motors to dynamically optimize the detection efficiency for a given spatial point.

The planar sector comprises: a collimator 2a configured to allow the secondary particle in selected regions of interest with a gap, wherein the collimator 2a is made of a dense material; a sensitive array detector 2b configured to detect the secondary particle that pass through the collimator 2a; a readout electronic and communication module_2 2c comprising a least one circuit capable of reading the sensitive array detector 2b, wherein the readout electronic and communication module_2 2c communicates with the central module 3; and a mechanical unit 2d configured to package and adjust the collimator 2a positions in the external array detector 2.

The collimator 2a is made of a dense material which refers to lead, tungsten, metal alloys with densities higher than 7. g.cm-3 that can cause significant attenuation of the high energy gamma photons, or their combination thereof.

As shown in FIG. 18, the FIG. is related to the external detector 2, and describes the spatial resolution achieved using the single module described in FIG. 7 for one particular configuration. Positional resolution of the located gamma source as a function of collimator width simulated with a 990 keV for three different collimator gap values w1=1 mm, w1=2.5 mm and w1=5 mm In this setup D1=300 mm and D2=330 mm

TABLE 2
Spatial resolution and the region of interest (ROI) for
localizing a point source on the axis of interest for three
different collimator gaps. The photo-peak detection efficiency
values for low energy and high energy gamma are shown.
The entire setup was simulated on GATE/Geant4.

| | | Positional | Efficiency | Efficiency |
|---|---|---|---|---|
| Collimator | ROI | resolution | (sector⁻¹) | (sector⁻¹) |
| gap w1 mm | w2 mm | mm (FWHM) | (at 990 keV) | (at 6.13 MeV) |
| 1 | 2.8 | 2.6 | 8.3 × 10⁻⁶ | 5.4 × 10⁻⁶ |
| 2.5 | 7.0 | 5.6 | 2.2 × 10⁻⁵ | 1.1 × 10⁻⁵ |
| 5 | 14.1 | 11.7 | 4.4 × 10⁻⁴ | 3.1 × 10⁻⁴ |

Table 2 is related to FIG. 18 and an illustration of the performance of external detector 2. The table summarizes the spatial resolution and the region of interest (ROI) for localizing a point source on the axis of interest for three different collimator gaps. These values were obtained for clinically relevant distances where D1=300 mm and D2=330 mm. The photo-peak detection efficiency values for low energy (990 keV) and high energy (6.13 MeV) gamma are shown. The entire setup was simulated on GATE/Geant4.

As shown in FIG. 8, the location for placing the multiple sectors is obtained by first rotating the primary sector about the beam axis and then translating along the beam axis. To increase the detection efficiency, there are multiple modules within each planar sector of the external detector device 2 to detect the secondary particle during the operation time of the present invention. Each of the planar sectors has its primary sub-module which is placed in a manner that the collimators 2a allow the photon form a narrow angular window.

As used herein,” sub-module” means that the external detector device 2 in the present invention which is modularized. Each sub-module comprises at least one collimator 2a and sensitive array detector 2b.

As used herein,” the axis of the sub-module” that is defined to be the angular bisector of this collimating angular window.

Inorganic scintillating crystals for both active-collimation and shielding are used for active collimation. The sensitive array detector 2b comprises a dedicated scintillating crystal such as LYSO, LaBr3, CLYC, CLLB or other inorganic scintillating crystals for converting the gamma into visible light with a high attenuation and a low value of energy resolution. The sensitive array detector 2b also comprises photo detectors to read the scintillation light output. The geometry of the module is described in FIG. 7.

There is a formula(I) below and it is relation between parameters of collimator gap, the separation distances and the crystal sizes that can be chosen or adjusted.

$$w 2 = w 1 ⨯ ( 2 \frac{D 1}{D 2} + 1 ) \qquad ( I )$$

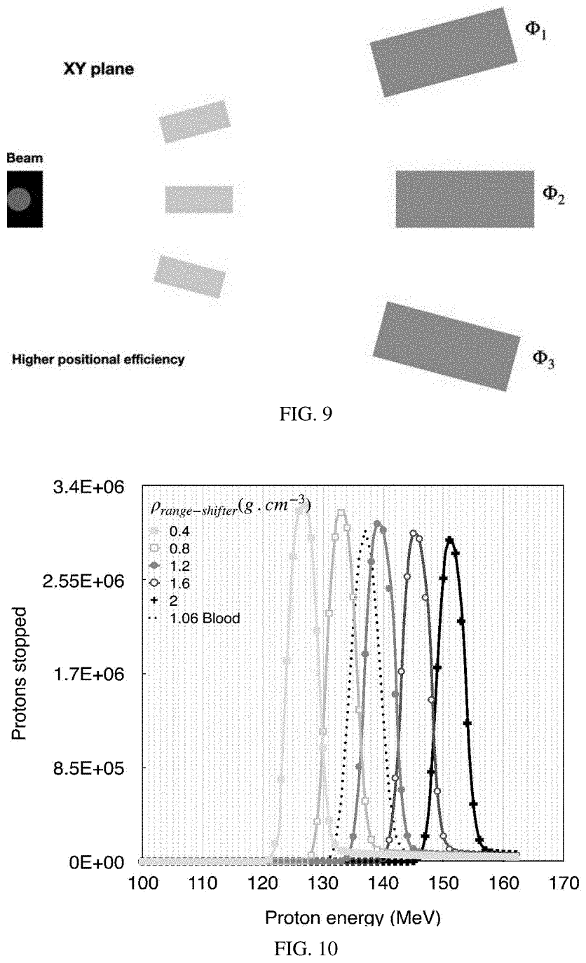

Because the external detector device 2 comprised multiple sub-modules which contain collimators 2a and sensitive array detector 2b, the gap of every pair of collimators 2a needs to adjust according to the status of the object and its range is 0.1 to 10 mm. The gap of every pair of flat and parallel collimators is 0.1 to 10 mm. In the preferred embodiment, based on an optimization, the recommended values are: D1=30 cm, D2=33 cm, collimation gap w1=1-5 mm, and a size of 30 mm×40 mm sections and 50-100 mm length for the crystal forming the sensitive array detector 2b. Each module once designed is repeated in a geometrically calculable manner to achieve the remaining part of the sensitive array detector 2b. FIG. 9 shows multiple sub-modules in each sector are placed in a rotational symmetry along with azimuth with respect to the primary sub-module.

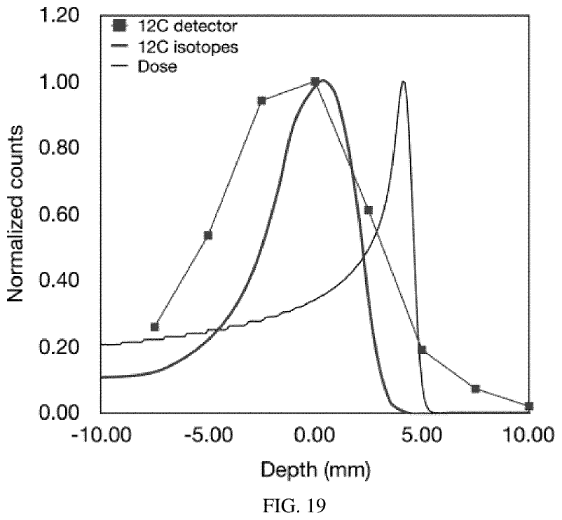

As shown in FIG. 19, it describes the performance of the external detector 2 constructed using three modules and eight sectors separated with a 15-degree angle. Each module is built with w1=2.5 mm, D1=300 mm and D2=330 mm The sensitive detector is a LYSO detector with 70 mm×40 mm×100 mm. The longest dimension 100 mm is along the axial direction, 70 mm is the thickness along the radial direction, and 40 mm is the height along theta direction. In a simulation of a water target irradiated with 40 MeV protons, the Intensity of gamma lines originating from 16O(p,x)12C isotopes as registered by eight different sectors along the detector model. Consecutive sectors are separated axially by 2.5 mm. The original depth distribution of the isotopes is shown for comparison

Each module has a fixed frame of reference connected via a motor that allows small angular rotation. By individually controlling the rotational angle of the module about the initial value, the sensitive array detector 2b can be made to focuses on the emitted gamma nearer or farther from the original focal point (D1+D2). Each sector is further mounted on a linear motor stage that allows the various sectors to be configured in one embodiment to focuses on the same point allowing a maximal efficiency in a region of interest.

In another embodiment, each sector can be positioned to focuses on the different points along the beam path thereby allowing a larger field of view in identifying the region of interest for gamma emission.

The external detector device 2 comprises a positioning unit 2e that is used to adjust the global position and orientation of the sensitive detection system. Once the initial position of the external detector device 2 is set with respect to the laser beam, the sensitive array detector 2b is free to translate and keep track of its position. The external detector device 2 accepts control signals from the central module 3 that depend on the treatment plan and beam delivery parameters that the central module 3 receives from the beam control device 5.

Central Module 3

The central module 3 is a system that interacts with the other systems to supply the power, collect information, process the signals, perform computation and communicate pertinent control signals.

In one embodiment of the present invention, the central module 3 is equipped with a software capable of resolving gamma energy from the array detector 1c, external detector device 2.

The electronic and communication module_1 1d receives/transmits signals from the implant module 1, the external detector device 2, the positioning system 4, and the beam control device 5. In one embodiment of the present invention, the processing unit retrieves the CT image from the disk. Calculates the patient position from the positional module 4 and maps the CT image to the current position of the patient and the implant module 1 and the external detector device 2. Communicates a set of scanning beam parameters of position, energy and current to the beam control device 5.

Processes the array detector 1c data from the implant module 1 and the sensitive array detector 2b data from the external detector device 2 to obtain the signal strengths from various channels from the corresponding detectors. This information is compared to the expectation values of the detector signals pre-calculated in accordance to the parameters issued to the beam control device 5. A look-up table 3c is generated prior to the irradiation of the target for a set of pre-calculated values of a set of beam positions, energies, and the beam currents for different cases of range shifters introduced through a Monte Carlo Simulation framework.

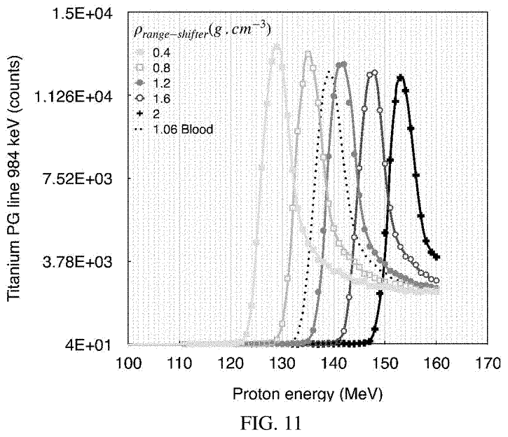

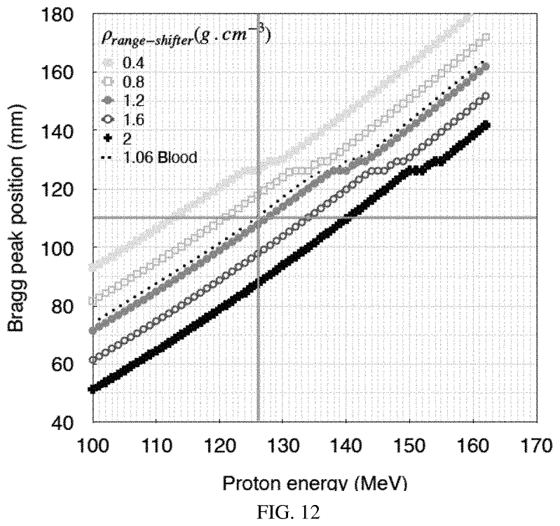

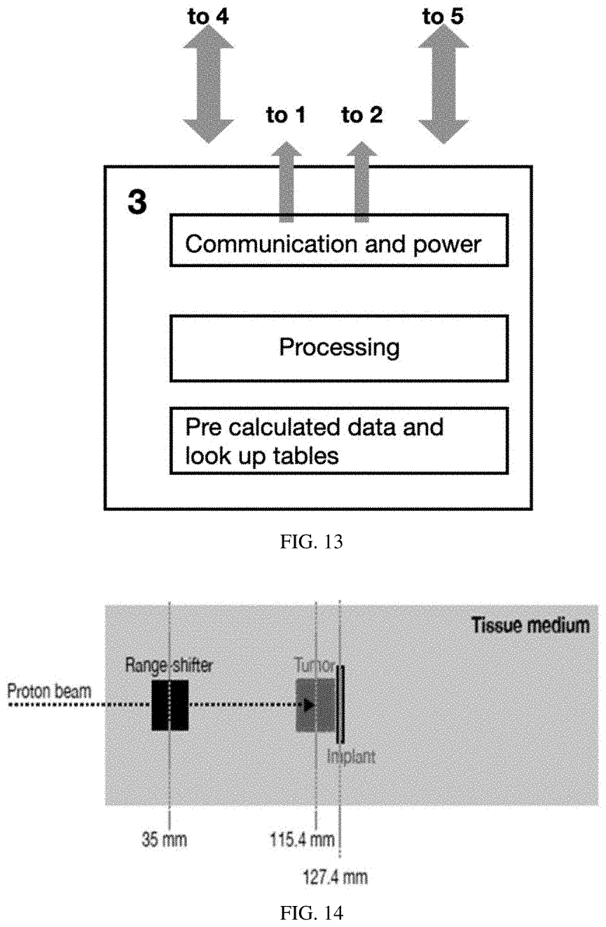

As shown in FIG. 10 to FIG. 12 of the present invention, the flux distribution of protons and gamma at the target/detector in the implant module 1 for a range of input proton energies at various values of ranger shifter material simulated upstream (FIG. 14). The peak values simulated are compared with the measured ones to identify the range shifter that gives a best match of the peak value. The curve from FIG. 12 for the corresponding matched material will be helpful in identify the actual position of the Bragg peak at the treatment energy. The central module 3 accepts signals and compares with pre-determined simulations to estimate a beam energy correction. The look-up table 3c is populated with the expected detector signal values from the implant detection unit 1a, the array detector 1c and the external detector device 2, and the expected dose deposition at the entrance and exit of the patient treatment volume, the expected dose deposition at the implant detection unit 1a and the implanted material 1b. The deviations in the signal are compared to pre-calculated scenarios from the look-up table 3c. The necessary adjustment in the beam energy is calculated accordingly. This value is communicated to the central module 3. During the treatment, the central module 3 issues a new position to the external detector device 2 to adjust the focus to the tumor region.

The central module 3 comprises: (a) an electronic and communication module_3 3a configured to receives/transmits signals from the implant module 1, the external detector device 2, the positional system 4 and the beam control device 5; (b) processing unit 3b configured to integrate the signals from the electronic and communication module_3 3a; and (c) look-up table 3c generated prior to the irradiation of the target for a set of pre-calculated values, wherein the look-up table 3c is used for estimating a beam correction.

Positioning Unit 4

The objective of the positioning module 4 is to obtain the patient position in order to localize the gamma image from the implant module 1 and the external detector device 2 with respect to the patient CT image. Normally, the medical physicists in the therapy center use the existing methods in the treatment facility to fix the patient position relative to the treatment couch and used markers on the patient body to align the isocenter using laser beam in the gantry. In this scenario, the external detector device 2 can be aligned using the aforementioned laser beam.

Alternatively, an orthogonal X-ray system, or a resistive mat 7 along with an external marker for breath monitoring can be used for this purpose and the information relayed to the positioning module 4.

In a preferred embodiment, the resistive-mat 7 locates the patient position based on one point for the head, two points for the shoulder blades, two points for the buttocks, two points for the heels. By using this information, the relevant points can be aligned with a pre-existing CT image to lock the patient coordinates digitally. An external sensor will be monitored by a camera on positioning system 4 that allows the observation of the breathing cycles. This information can be relayed through the central module 3 to the beam control device 5. The breath information allows the scanning pencil beam to adjust the range of positions about the central value in sync with the breathing pattern. The external sensor for scanning the patient motion can be implemented in the embodiment (FIG. 3) used for tumors in the torso region of the patient.

The particle induced radiography system can provide flexible and higher accuracy proton beam to treat patients in different environment. A deviation in the location of the Bragg peak during the treatment when compared to the treatment-plan can place the sensitive organs at risk. As shown in FIG. 14, the diagram of the present invention shows that such a small volume of 20×20× 20 mm3 is used to model a range-shifter in the form of a variable density. A beam energy scan is performed for a low dose, and the detected secondary particle information obtained will be useful to assess the fidelity of the treatment plan and re-creating a new treatment plan instantaneously.

The proton flux at the implant, the secondary gamma flux due to the implant, and the Bragg peak position are recorded for several proton energies between 100-160 MeV. The targeted energy is 126 MeV to be delivered in the center of the tumor at 115 mm as seen in FIG. 12. For this simulation, the tumor and the tissue are modeled with a density of 1.06 g/cm-3, while the implant has a density of 4.5 g/cm-3.

In the ideal case, the most important thing is that the dose of the beam delivered during therapy process needs to match the treatment-planning dose. For example, if the operators expect to see a peak in the proton flux measured by the implant at a proton energy of 137 MeV. By identifying which proton energy results in the highest proton flux inside the implant, the corresponding range shifter density can be identified. For example, if the peak flux is seen at 135 MeV proton energy, the range-shifter will be tagged as 0.8 g/cm-3. By looking in the FIG. 10 for this case of 0.8 g/cm⁻³, the correct proton energy for the tumor (115 mm from the entrance) can be identified as 121 MeV requesting the beamline to reduce the original value by 5 MeV. A similar approach can be adopted to monitor the counts of the characteristic gamma photons from the implanted marker such as “titanium”.

3D Imaging System

The 3D imaging system comprises a particle radiation source device_2 8 configured to provide a beam; a beam control device_2 9 configured to adjust the particle radiation source device_2 8 and control the beam energy; an external detector device_2 10 configured to receive the secondary particles emitted by the target object after the beam irradiation; a positioning module_2 11 configured to obtain the object position information to localize the image from the external detector device_2 10; and a central module_2 12 configured to enable conversion of the secondary particle into elements by accessing the secondary particle production cross sectional information along with reconstruction techniques.

The 3D imaging system comprising: (a) a particle radiation source device_2 configured to irradiate a pencil beam at various positions on the target material; (b) a beam control device_2 configured to adjust the particle radiation source device_2 and control beam energy to deliver the beams at different positions; (c) an external detector device_2 configured to receive the secondary particles emitted by a target object during and after the beam irradiation, which can be synchronized to optionally move with the beam position to remain in focus on the beam axis; (d) a positioning module_2 configured to obtain object position information to localize the image from the external detector device_2; and (e) a central module_2 configured to enable conversion of the secondary particle into elements by accessing the secondary particle production cross sectional information along with reconstruction techniques.

The reconstruction may be performed either through analytical techniques such as filtered back projection, or statistical techniques such as the Maximum Likelihood Expectation Maximization method (MLEM), or through training a neural network on the entire system performance, on various target materials, and providing the experimental conditions as prior, to achieve a direct reconstruction of the original object's composition. Known target materials and compositions will be provided as labeled during the training phase in this implementation of reconstruction using neural networks. Conditional GANs, other variants of GANs can be examples of such reconstruction.

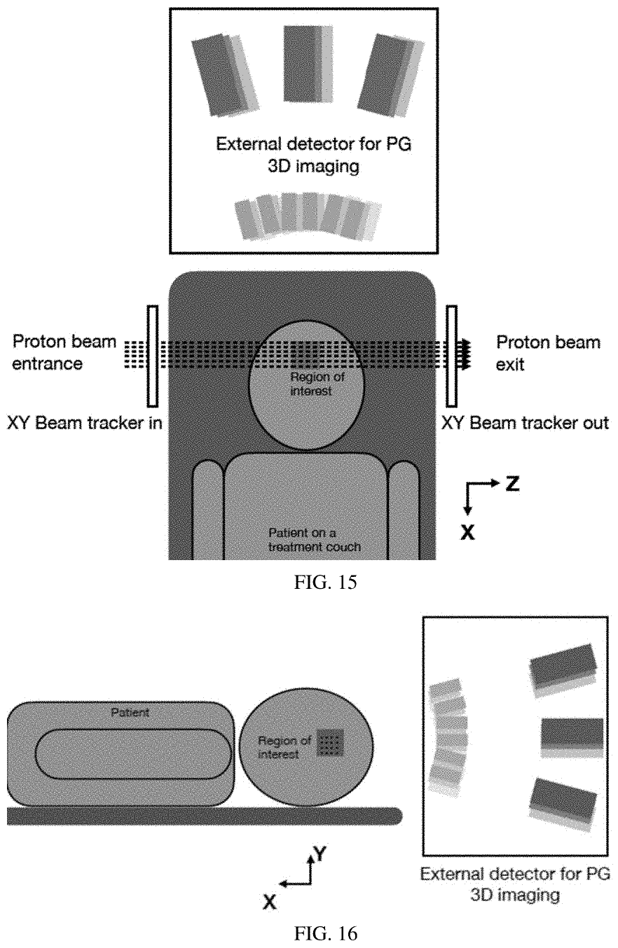

As FIGS. 15 and 16 shown, the 3D imaging system uses the beam to observe the region of interest. In this 3D imaging system, the external detector device_2 10 comprises sub-module. Each sub-module consists of at least one collimator_2 10a and followed by a sensitive array detector_2 10b. The sensitive array detector_2 10b should be used in tandem with collimated particle beam to perform a 2D scan of several points at a time. The beam delivery position, energy information and the order of scanning need to be synchronized with the central module_2 12.

According to the actual condition, the operator could arrange the multiple planar sectors of the external detector device_2 10 in a way that each sector focusses on the same spatial point along the beam axis. Alternatively, the multiple plan sectors of the external detector device_2 10 can each be arranged to focus on a different spatial point along the beam axis.

The operators could use electro-mechanical motors to linearly shift each sector of the external detector device_2 10 to dynamically optimize the detection efficiency for a given spatial point in relation to the number of spatial points simultaneously detected.

Each sub-module within the sector of the external detector device_2 10 can be further rotated in a small range of angles to adjust the precision of the focus achieved using a rotational motor for each sub module.

To avoid exposing the patient to high dose of the particle beam, the proton energy used for imaging must be high enough to exit the patient or the target object with an energy higher than a few tens of MeV. As shown in FIG. 15, the boundaries of the XY scan should be sufficient to enclose the desired region of interest. The set of XY points for the beam irradiation will be planned beforehand and the information stored in the central module_2 12 will synchronously drive both the proton beam and the external detector device_2 10 to obtain the 1D images for each XY point. The sensitive array detector_2 10b will be positioned radially to the beam in a manner that enables it to focus on the region of interest.

For a given single XY position at which the beam is positioned, several points along the Z axis will be monitored. Due to the high level of collimation, the obtained image after performing a reconstruction will yield the 1D-prompt gamma spectrum. The beam will then move to the next chosen XY point and the process is repeated.

In a preferred embodiment, as shown in FIG. 15, the 3D imaging system further comprises XY trackers which can be added at the entrance and exit of the protons into the patient or the target. The XY trackers are made of pixelated ionization chambers or solid state pixel detectors. Such trackers will help to reject events that are scattered significantly and hence achieve a high degree of collimation along the XY axes.

What is claimed is:

1. A particle induced radiography system comprising:
(a) a particle radiation source device configured to irradiate a beam;
(b) a beam control device configured to adjust the particle radiation source device to control the beam energy;
(c) an implant module configured to present a location of an object and to receive and detect the beam from the particle radiation source device, wherein the implant module comprises:
an implant detection unit configured to detect the number of particles from the beam,
at least one implanted material configured to interact with the beam that irradiated by the particle radiation source device to generate a secondary particle,
an array detector configured to detect the secondary particle, an electronic and communication module_1 configured to readout data from the array detector and to output the readout data, and an extension unit configured to provide for optical or mechanical tracking of the implant module;
(d) an external detector device configured to receive the data from the electronic and communication module_1 and detect the secondary particle which is generated from the implanted material to generate a signal and output to the central module for integrating;
(e) a positioning module configured to obtain location of the object in order to localize the image from the implant module 1 and the external detector device; and
(f) a central module configured to process the signals, perform computation and communicate pertinent control signals, wherein the central module receives and/or transmit signals from the implant module or the external detector device and transmits to other modules.

2. The particle induced radiography system of claim 1, wherein the secondary particle is gamma, electron, neutron, proton or prompt gamma.

3. The particle induced radiography system of claim 1, wherein the implant module further comprises a package configured to serve as a container for the implant module.

4. The particle induced radiography system of claim 1, wherein the array detector is an array of scintillating crystals silicon photomultiplier, avalanche photodiode, photomultiplier tube or other optically sensitive detector.

5. The particle induced radiography system of claim 1, wherein the external detector device consists of at least one sub-module within a planar sector.

6. The particle induced radiography system of claim 5, wherein the planar sector comprises:
(a) a collimator configured to allow the secondary particle in selected regions of interest with a gap, wherein the collimator is made of a dense material;
(b) a sensitive array detector configured to detect the secondary particle that pass through the collimator;
(c) a readout electronic and communication module_2 comprising a least one circuit capable of reading the sensitive array detector, wherein the readout electronic and communication module_2 communicates with the central module; and
(d) a mechanical unit configured to package and adjust the collimator positions in the external array detector.

7. The particle induced radiography system of claim 6, further comprises: positioning unit configured to adjust the global position and orientation.

8. The particle induced radiography system of claim 5, wherein the at least one sub-module is placed in a manner that a gap of pair of collimators allows the secondary particle from a narrow angular window to be detected.

9. The particle induced radiography system of claim 8, wherein the gap of every pair of collimators is 0.1 to 10 mm.

10. The particle induced radiography system of claim 1, wherein the central module comprises:
(a) an electronic and communication module_3 configured to receives/transmits signals from the implant module, the external detector device, the positional system and the beam control device;
(b) processing unit configured to integrate the signals from the electronic and communication module_3; and
(c) look-up table generated prior to the irradiation of the target for a set of pre-calculated values, wherein the look-up table is used for estimating a beam correction.

11. The particle induced radiography system of claim 1, wherein the central module accepts signals and compares with pre-determined simulations to estimate a beam energy correction.

12. The particle induced radiography system of claim 1, wherein the central module is configured to enable the conversion of the gamma into dose profiles.

13. The particle induced radiography system of claim 1, wherein the positional module creates a digital map of the object based on the positional signal from a resistive mat attached to a treatment couch.

14. The particle induced radiography system of claim 1, wherein the object is a patient, organ, tissue, animal plant, or a non-biological material sample.

15. The 3D imaging system comprising:
(a) a particle radiation source device_2 configured to irradiate a beam;
(b) a beam control device_2 configured to adjust the particle radiation source device_2 and control beam energy;
(c) an external detector device_2 configured to receive the secondary particles emitted by a target object after the beam irradiation;
(d) a positioning module_2 configured to obtain object position information to localize the image from the external detector device_2; and
(e) a central module_2 configured to enable conversion of the secondary particle into elements by accessing the secondary particle production cross sectional information along with reconstruction techniques.

16. The 3D imaging system of claim 15, wherein the external detector device_2 consists of at least one sub-module within the planar sector.

17. The 3D imaging system of claim 15, wherein the external detector device_2 contains multiple sub-modules within the same planar sector.

18. The 3D imaging system of claim 15, wherein the external detector device_2 has multiple planar sectors.

19. The 3D imaging system of claim 15, wherein the position information includes the information of the target object and the external detector device_2 which are obtained through external imaging techniques.

20. The 3D imaging system of claim 15, wherein the sub-module comprises a collimator_2 and a sensitive array detector_2.
